(19) United States
(12) Patent Application Publication
(10) Pub. No.: US 2016/0246944 A1
(43) Pub. Date: Aug. 25, 2016

(54) METHOD AND SYSTEM FOR TREATING PAIN OF USERS

(71) Applicants: LAKSHYA JAIN, LOS ALTOS, CA (US); PRIYA BISARYA, SAN DIEGO, CA (US)

(72) Inventors: LAKSHYA JAIN, LOS ALTOS, CA (US); PRIYA BISARYA, SAN DIEGO, CA (US)

(21) Appl. No.: 14/629,471

(22) Filed: Feb. 23, 2015

(51) Int. Cl. G06F 19/00; A61N 2/02; A61N 2/00; A61H 39/08; A61N 7/02

(52) U.S. Cl. CPC G06F 19/3481; G06F 19/3456; G06F 19/322; A61H 39/086; A61N 7/02; A61N 2/004; A61N 2/002; A61N 2/02; A61H 2201/0221; A61H 2230/045; A61H 2230/065; A61H 2230/655; A61H 2230/405; A61H 2230/305; A61H 2230/208; A61H 2230/105; A61H 2230/605; A61H 2201/10; A61N 2007/0026

(57) ABSTRACT

The present disclosure provides a method and system for treating pain of one or more users. The method includes associating one or more sensors with the one or more users, tracking the intensity of pain of the one or more users, generating a plurality of profiles for each of the one or more users and facilitating treatment of the pain of the one or more users by utilizing the generated plurality of profiles. The one or more sensors measure one or more bio-markers associated with the one or more users. The tracking is performed by enabling interaction of one or more devices with the one or more users. The plurality of profiles is generated by the associating and the tracking of the intensity of the pain of the one or more users. The facilitating is based on one or more pre-determined criteria.

### TECHNICAL FIELD

The present invention relates to treating pain of users and, in particular, relates to treating the pain of the users by utilizing medical devices.

### BACKGROUND

Pain is an unpleasant sensory and emotional experience associated with actual or potential tissue damage. In fact, the pain is a stressor and environment challenge that requires the organism to respond. It is a specific emotion, caused by a stimulus that reflects homeostatic behavioral drive, similar to temperature, itching, hunger, thirst and the like. It may be categorized according to various factors, including type of damage, time for healing and the like. On the basis of healing, the pain can be categorized as a chronic pain and an acute pain. The chronic pain lasts for a longer time as compared to the acute pain. However, both the chronic pain and the acute pain are extremely important problems leading to loss of working capabilities, financial resources and the like.

Experience of the pain varies from person to person due to inter-individual variability. Moreover, intensity of the pain varies from cause to cause in the individual. Thus, pain management is an extremely important issue. Various factors, directly or indirectly, contributes in controlling the pain. For example, biological factors (for e.g., sex, genetics and the like), psychological factors (for e.g., mood, attention, distraction and the like), social factors (for e.g., marital status, social support and the like) and the like can significantly modulate intensity as well as unpleasantness caused by the pain.

Presently, it is known that functional interactions exist between systems predicting the pain and systems modulating perception of the pain. However, presently, there is no adequate method and system that studies these relationships and the interactions to guide appropriate treatment, care, lifestyle and the like for healthy response in individuals with respect to the pain. Moreover, the current methods and systems reduce the pain not in case of all the individuals. Further, these methods and systems do not reduce/treat the pain effectively in the individuals.

In light of the above stated discussion, there is a need for a method and system that overcomes the above stated disadvantages.

### SUMMARY

In an aspect of the present disclosure, a computer-implemented method for treating pain of one or more users is provided. The method includes associating, with a processor, one or more sensors with the one or more users; tracking, with the processor, intensity of pain of the one or more users; generating, with the processor, a plurality of profiles for each of the one or more users and facilitating, with the processor, treatment of the pain of the one or more users by utilizing the generated plurality of profiles. The one or more sensors measure one or more bio-markers associated with the one or more users in real time. The tracking is performed by enabling interaction of one or more devices with the one or more users. The plurality of profiles is generated by the associating and the tracking of the intensity of the pain of the one or more users. The facilitating is based on one or more pre-determined criteria.

In an embodiment of the present disclosure, the computer-implemented method further includes storing, with the processor, the one or more bio-markers associated with each of the one or more users and the generated plurality of profiles.

In an embodiment of the present disclosure, the plurality of profiles includes one or more pain profiles, one or more side-effect profiles and one or more treatment profiles and the like.

In an embodiment of the present disclosure, the one or more sensors includes a ballistocardiogram, an impedance cardiography, a dispersion based electrocardiography, a respiration sensor, an emotion detector and the like.

In an embodiment of the present disclosure, the one or more bio-markers associated with each of the one or more users includes heart rate (HR), heart rate variability (HRV), skin conductance, respiration information, blood pressure, photoplethysmography (PPG), oxygen saturation, electrocardiography (ECG), electroencephalography (EEG), muscle activity (EMG), restlessness and the like.

In an embodiment of the present disclosure, the one or more devices includes one or more transcutaneous electrical nerve simulation devices, one or more acupuncture devices, one or more devices providing ultrasound based pain relief, one or more spinal stimulators and the like.

In an embodiment of the present disclosure, the computer-implemented method further includes assessing, with the processor, the intensity of the pain of the one or more users by viewing the intensity of the pain on displays of the one or more devices.

In an embodiment of the present disclosure, a pre-determined criterion of the one or more pre-determined criteria includes throbbing of pain area on body of each of the one or more users. The throbbing enables application of symmetrical pulses of fixed frequency to reduce the pain.

In another embodiment of the present disclosure, a pre-determined criterion of the one or more pre-determined criteria includes application of at least one of heat, pressure and laser light on the pain area on the body of each of the one or more users.

In yet another embodiment of the present disclosure, a pre-determined criterion of the one or more pre-determined criteria includes electromagnetically heating of the pain area on the body of each of the one or more users by utilizing ultrasonic radiations.

In another aspect of the present disclosure, a computer system is provided. The computer system includes a non-transitory computer readable medium storing a computer readable program; the computer readable program when executed on a computer causes the computer to perform steps. The steps include associating one or more sensors with one or more users, tracking intensity of pain of the one or more users, generating a plurality of profiles for each of the one or more users and facilitating treatment of the pain of the one or more users by utilizing the generated plurality of profiles. The one or more sensors measure one or more bio-markers associated with the one or more users in real time. The tracking is performed by enabling interaction of one or more devices with the one or more users. The plurality of profiles is generated by the associating and the tracking of the intensity of the pain of the one or more users. The facilitating is based on one or more pre-determined criteria.

In an embodiment of the present disclosure, the computer readable program when executed on the computer causes the computer to perform a further step of storing the one or more bio-markers associated with each of the one or more users and the generated plurality of profiles.

In yet another aspect of the present disclosure, a system for treating pain of one or more users is provided. The system includes one or more devices and a communication device. The one or more devices treat the pain of the one or more users. The one or more devices includes one or more transcutaneous electrical nerve simulation devices, one or more acupuncture, or electroacupuncture or acupuncture-like devices, one or more devices providing ultrasound based pain relief, one or more spinal stimulators, electrical massager, transcranial magnetic stimulation, pulsed-electromagnetic field based devices, and the like. The communication device includes a pain monitoring application. Further, the pain monitoring application includes an associating engine in a processor, to associate one or more sensors with the one or more users; a tracking module in the processor, to track intensity of pain of the one or more users; a generating module in the processor, to generate a plurality of profiles for each of the one or more users; a treatment module in the processor, to treat the pain of the one or more users by utilizing the plurality of profiles and a database in the processor, to store the one or more bio-markers associated with each of the one or more users and the generated plurality of profiles. The one or more sensors measure one or more bio-markers associated with the one or more users in real time. The tracking is performed by enabling interaction of the one or more devices with the one or more users. The plurality of profiles is generated by the associating and the tracking of the intensity of the pain of the one or more users. The facilitating is based on one or more pre-determined criteria.

In an embodiment of the present disclosure, the system further includes an application server to run the pain monitoring application.

In an embodiment of the present disclosure, the plurality of profiles includes one or more pain profiles, one or more side-effect profiles, one or more treatment profiles and the like.

In an embodiment of the present disclosure, the one or more sensors includes a ballistocardiogram, an impedance cardiography, a dispersion based electrocardiography, a respiration sensor, an emotion detector and the like.

In an embodiment of the present disclosure, the one or more bio-markers associated with each of the one or more users includes at least one of heart rate (HR), heart rate variability (HRV), skin conductance, respiration information, blood pressure, photoplethysmography (PPG), oxygen saturation, electrocardiography (ECG), electroencephalography (EEG), muscle activity (EMG), restlessness and the like. Please add the long list of sensors that we have been adding in ALL the patent drafts during the revision of these drafts.

In an embodiment of the present disclosure, a pre-determined criterion of the one or more pre-determined criteria includes throbbing of pain area on body of each of the one or more users. The throbbing enables application of symmetrical pulses of fixed frequency to reduce the pain.

In another embodiment of the present disclosure, a pre-determined criterion of the one or more pre-determined criteria includes application of at least one of heat, pressure and laser light on the pain area on the body of each of the one or more users.

In yet another embodiment of the present disclosure, a pre-determined criterion of the one or more pre-determined criteria includes electromagnetically heating of the pain area on the body of each of the one or more users by utilizing ultrasonic radiations.

### BRIEF DESCRIPTION OF THE FIGURES

Having thus described the invention in general terms, reference will now be made to the accompanying drawings, which are not necessarily drawn to scale, and wherein:

FIG. 1 illustrates a system showing an interaction among various components for treating pain of users, in accordance with various embodiments of the present disclosure;

FIG. 2 illustrates a block diagram of a communication device, in accordance with various embodiments of the present disclosure; and

FIG. 3 is a flowchart for treating the pain of the users, in accordance with the various embodiments of the present disclosure.

### DETAILED DESCRIPTION

It should be noted that the terms “first”, “second”, and the like, herein do not denote any order, quantity, or importance, but rather are used to distinguish one element from another. Further, the terms “a” and “an” herein do not denote a limitation of quantity, but rather denote the presence of at least one of the referenced item.

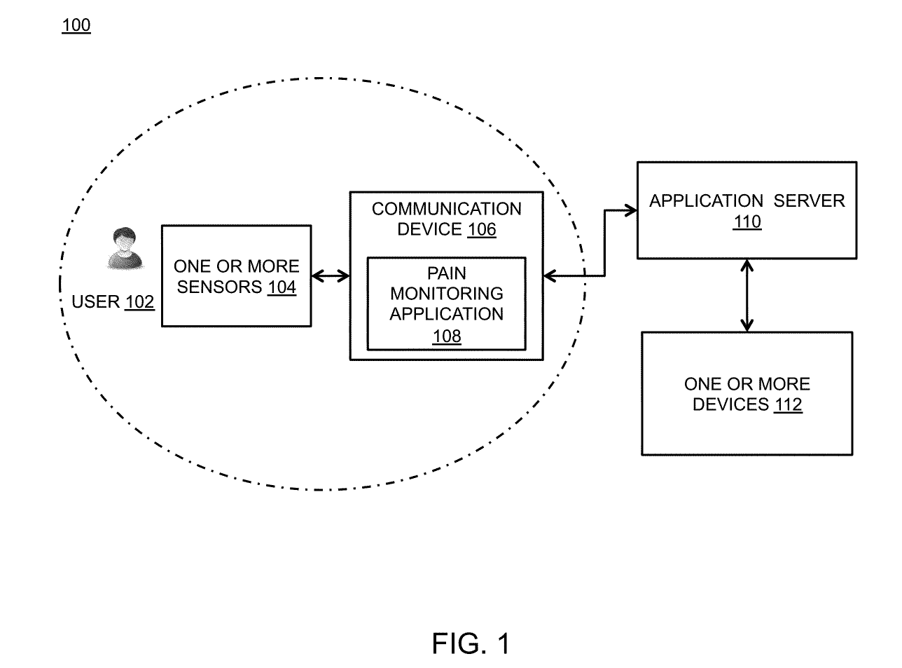

FIG. 1 illustrates a system 100 showing interaction among various components for treating pain of users, in accordance with various embodiments of the present disclosure. The system 100 includes one or more sensors 104 and a communication device 106 associated with a user 102. Examples of the communication device 106 include but may not be limited to mobile phone, laptop, desktop computer, PDA and the like. The communication device 106 executes a pain monitoring application 108. The pain monitoring application 108 monitors the pain of the user 102 and allows tailoring of treatments accordingly. The pain monitoring application 108 communicates with an application server 110 via a network. The application server 110 is associated with one or more devices 112.

The one or more sensors 104 fetches one or more bio-markers associated with the user 102 in real time including systolic contraction, systemic resistance, cardiac output (for example, heart rate, heart rate variability (HRV), blood flow, blood pressure, movements due to shifts in central blood mass, myocardial electrophysiological responses and the like), respiration information, emotions, skin conductance, photoplethysmography (PPG), oxygen saturation, electrocardiography (ECG), electroencephalography (EEG), muscle activity (EMG), accelerometer, EOG, temperature, blood glucose and the like. In an embodiment of the present disclosure, the one or more sensors 104 may be embedded in a wearable component or a device held close to the user 102. The one or more sensors 104 include but may not be limited to a ballistocardiogram, an impedance cardiography, a dispersion based electrocardiography, a respiration sensor and an emotion detector.

Variability in the one or more bio-markers associated with the user 102 corresponds to autonomic reactions. Examples of the variability in the one or more bio-markers include but may not be limited to systolic contraction, systemic resistance, cardiac output (for example, heart rate, heart rate variability (HRV), blood flow, blood pressure, movements due to shifts in central blood mass, myocardial electrophysiological responses and the like), respiration information, emotions, skin conductance, photoplethysmography (PPG), oxygen saturation, electrocardiography (ECG), electroencephalography (EEG), muscle activity (EMG), accelerometer, EOG, temperature, blood glucose, 3-axis accelerometer, 3-axis gyroscope and the like. In an embodiment, the accelerometers show greater movement as the user 102 becomes more restless under pain. These movements are picked up sensitively by the 3-axis accelerometer but may not be picked up as accurately by naked human eye. The angular part of movement is picked by the 3-axis gyroscope. For example, a sensor of the one or more sensors 104 measures the heart rate, the heart rate variability (HRV), the blood flow and the blood pressure of a user X and a sensor of the one or more sensors 104 measures the respiration information and oxygen saturation of a user Y.

These symptoms (the autonomic reactions) are analyzed by the pain monitoring application 108 to analyze location and intensity of the pain in body of the user 102. For example, a noticeable and progressively increasing change in certain dimensions of EEG reflects increasing pain which is measured by the one or more sensors 104. Further, the one or more devices 112 track the intensity of pain of the user 102. The tracking is performed by enabling interaction of the one or more devices 112 with the user 102. The one or more devices 112 reduces/treats the pain of the user 102. The one or more devices 112 include one or more transcutaneous electrical nerve simulation devices, one or more acupuncture devices, one or more devices providing ultrasound based pain relief, one or more spinal stimulators or any other device known in the art which is capable of tracking and reducing the pain of the user 102.

In an embodiment of the present disclosure, each of the one or more devices 112 has a display. A display of the device of one or more devices 112 show when the device has reached its optimal use at a certain location in the body. In other words, if the user 102 is finding comfort in that area, the biomarkers are becoming better. In addition, the display may also indicate that it is either time to move to a new area on the body or time to stop. And, if the biomarkers are not improving then may be it is time to try something new or may be one is working on the wrong part of the body of the user 102.

Extending the above stated example, the application server 110 associates the plurality of profiles of the user X with a transcutaneous electrical nerve simulation device and the plurality of profiles of the user Y with an acupuncture device. Then, the application server 110 enables an interaction of the transcutaneous electrical nerve simulation device with the user X and the acupuncture device with the user Y.

Further, the pain monitoring application 108 generates a plurality of profiles for the user 102 by assessing the intensity of the pain of the user 102 from the one or more bio-markers by utilizing the one or more sensors 104. The plurality of profiles include one or more pain profiles associated with the user 102, one or more side-effect profiles associated with the user 102, one or more treatment profiles associated with the user 102 or any other type of profile known in the art that is associated with the treatment of the pain of the user 102.

The application server 110 runs the pain monitoring application 108. Further, the application server 110 facilitates treatment of the pain of the user 102 by utilizing the plurality of profiles. A physician may assess the intensity of the pain of the user 102 by viewing the intensity of the pain on displays of the one or more devices 112.

Continuing with the above stated example, the pain monitoring application 108 generates the plurality of profiles for the user X and the user Y. Further, the application server 110 facilitates treatment of the pain of the user X by utilizing his/her plurality of profiles and his/her interaction with the device. Furthermore, a physician P may adjust parameters including amplitude, voltage, frequency and period of signal delivered by using the device. Also, the application server 110 facilitates treatment of the pain of the user Y by utilizing his/her plurality of profiles and his/her interaction with the acupuncture device.

It may be noted that in FIG. 1, the application server 110 facilitates the treatment of the pain of the user 102; however those skilled in the art would appreciate that the application server 110 may facilitate the treatment of the pain of more than one user. It may also be noted that the user 102 is associated with the communication device 106; however those skilled in the art would appreciate that the user 102 may be associated with more than one communication devices having installed the pain monitoring application.

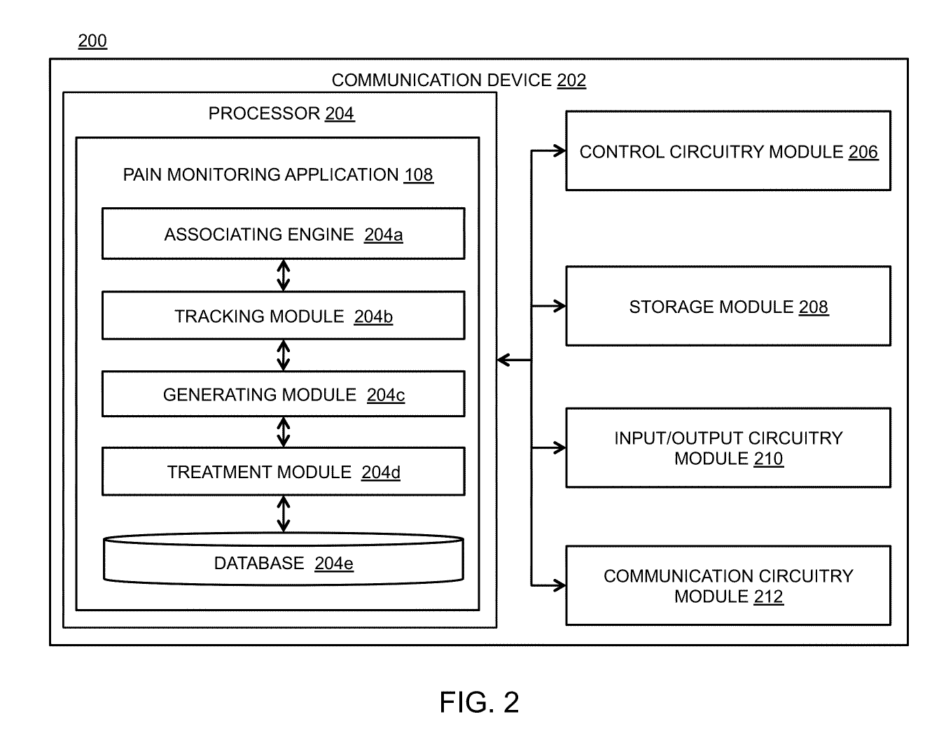

FIG. 2 illustrates a block diagram of the communication device 202, in accordance with various embodiments of the present disclosure. It may be noted that to explain the system elements of FIG. 2, references will be made to the system elements of FIG. 1. The communication device 202 includes a processor 204, a control circuitry module 206, a storage module 208, an input/output circuitry module 210 and a communication circuitry module 212. Further, the processer 204 includes an associating engine 204a, a tracking module 204b, a generating module 204c, a treatment module 204d and a database 204e. The above stated components of the processor 204 enable the working of the pain monitoring application 108.

The associating engine 204a associates the one or more sensors 104 with the user 102. The one or more sensors measure the one or more bio-markers associated with the user 102 in the real time. The one or more bio-markers that helps in assessing the intensity of the pain of the user 102 include the heart rate (HR), the heart rate variability (HRV), the skin conductance, the respiration information, the blood pressure, the photoplethysmography (PPG), the oxygen saturation, the electrocardiography (ECG), the electroencephalography (EEG), the muscle activity (EMG), restlessness and the like. Moreover, the one or more sensors 104 that measures the one or more bio-markers include a ballistocardiogram, an impedance cardiography, a dispersion based electrocardiography, a respiration sensor, an emotion detector and the like (as illustrated in detailed description of FIG. 1).

The tracking module 204b tracks the intensity of pain of the user 102. The tracking is performed by enabling interaction of the one or more devices 112 with the user 102. The one or more devices 112 includes the one or more transcutaneous electrical nerve simulation devices, the one or more acupuncture devices, the one or more devices providing ultrasound based pain relief, the one or more spinal stimulators or any other device capable of interacting with the user 102 for treating the pain of the user 102. Further, the one or more devices 112 interact with the user 102 for reducing the intensity of the pain of the user 102.

The generating module 204c generates the plurality of profiles for the user 102 by analyzing the intensity of the pain experienced by the user 102. The intensity is analyzed for each increment between former pain level and next pain level and is based on the associating of the one or more sensors 104 with the user 102 and the tracking the intensity of pain of the user 102. The plurality of profiles includes the one or more pain profiles, the one or more side-effect profiles, the one or more treatment profiles and the like.

The treatment module 204d facilitates the treatment of the pain of the user 102 by utilizing the plurality of profiles. The facilitation of the treatment is based on one or more pre-determined criteria. In an embodiment of the present disclosure, a pre-determined criterion of the one or more pre-determined criteria includes throbbing of pain area on body of the user 102. The throbbing enables application of symmetrical pulses of fixed frequency to reduce the pain of the user 102. For example, a user X interacts with the transcutaneous electrical nerve stimulation (hereinafter ‘TENS’) to reduce/treat his/her pain. For treating the pain, the user X connects two electrodes of the TENS to his/her skin or the pain area of the body. When a current is delivered through the electrodes by the TENS to the pain area, the current stimulates nerves in an affected area and sends signals to the brain that blocks normal pain signals. In an embodiment, the electrical stimulation of the nerves may help the body to produce natural pain killers which may block perception of the pain. The physician may adjust the parameters including the amplitude, the voltage, the frequency and the period of signal delivered by using the TENS.

In another embodiment of the present disclosure, a pre-determined criterion of the one or more pre-determined criteria includes application of at least one of heat, pressure and laser light on the pain area of the body of the user 102. For example, the user X interacts with the acupuncture to reduce/treat his/her pain. The acupuncture involves penetration of thin needles into the pain area of the skin or the application of heat, pressure, or laser light into the pain area of the skin. The treatment of the pain using the acupuncture may alleviate some, but not all, kinds of pain.

In yet another embodiment of the present disclosure, a pre-determined criterion of the one or more pre-determined criteria includes electromagnetically heating of the pain area of the body of the user 102 by utilizing ultrasonic radiations. For example, the user X interacts with a device that provides ultrasound based pain relief. Such a device includes a transducer that is applied to the body and an electrical box that the transducer is attached with. The transducer is held over the area of the body that is being targeted for ultrasound diathermy therapy. The ultrasound produces sound waves and transmits the sound waves to the affected area through a hand-held probe using a conductive gel. This treatment promotes healing of some acute bone fractures, venous and pressure ulcers and surgical incisions. However, therapeutic ultrasound can cause burns or endothelial damage if applied incorrectly.

In an embodiment of the present disclosure, the user 102 may interact with the one or more spinal stimulators for treating chronic pain in their back and/or limbs. For example, the user X is suffering from a back pain, then he/she may treat the pain using the one or more spinal stimulators. This therapy reports a 50 to 70% reduction in overall pain as well as an increased ability to participate in normal family and work activities.

Going further, the database 204e stores the one or more bio-markers associated with the user 102 and the generated plurality of profiles.

In an embodiment of the present disclosure, a patient (the user 102) may be characterized at different levels including health behavioural, functional, physiological psychological and the like. For each of the patients belonging to these categories, various sub-categories can be created and machine learning can be applied on each of the levels. For example, a set of N users can be partitioned and arranged according to the sub-categories of the levels of characterization.

In an embodiment of the present disclosure, the categories of classification of the one or more users can be combined. For example, if there are 3 categories based on personality type classification system, and 5 categories based on BMI based classification system, then 15 categories can be created based upon combining both the classification systems. Further, each of these 15 categories can be broken into 3 more categories based on ayurvedic system, characterization based on Chinese medicines and the like.

In an embodiment of the present disclosure, one or more pre-determined scales are utilized to measure the intensity of pain experienced by the user 102. The one or more pre-determined scales may include but not be limited to a visual analog scale (VAS), a behavior pain scale (BPS), a descriptor differential scale (DDS), a dolorimeter pain index (DPI), a neck pain and disability scale, a physician defined scale and the like.

In an embodiment of the present disclosure, the intensity of the pain experienced by the user 102 is increased and measured several times on the user 102 to check consistency of, and improve upon the learned information with new observations. For example, the new observations are generated when the intensity of the pain experienced by the user 102 is increased again one or more times.

In an embodiment of the present disclosure, the method is repeated several times to obtain improved machine learning.

In an embodiment of the present disclosure, a baselining of the one or more users is utilized to treat a new user. The one or more users are most similar to the new user and the similarity is based on a pre-determined set of attributes and pain area. The new user is the person for whom the baselining has not been done.

In an embodiment of the present disclosure, a pre-determined set of grouping scales is used for determining personality type and personality group of the user 102. The pre-determined set of grouping scales includes at least one of tridimensional personality questionnaire test, dallas pain questionnaire, roland morris back pain questionnaire, wong baker faces pain rating scale, verbal numerical rating scale (VNRS), verbal descriptor scale (VDS) and brief pain inventory.

In an embodiment of the present disclosure, the personality group is based on determination of personality traits. The personality traits include at least one of harm avoidance (HA), reward dependence (RD) and novelty seeking (NS). In another embodiment of the present disclosure, the personality group is based on determination of personality traits using any other accepted method of determining such personality trait. This may include 5-factor model, International Personality Item Pool (IPIP) model, or any other model that has been accepted by experts in the field.

In an embodiment of the present disclosure, the generated plurality of profiles for the user 102 utilizes a pre-defined color coding based on the intensity and the location of the pain in body of the user 102. Intensity of colors increases proportionally with respect to the pain experienced by the user 102. Moreover, the intensity of colors may differentiate areas in the body of the user 102 that are most susceptible to the pain. Further, the intensity of colors may segregate affected or bruised areas in the body of the user 102 with the healthy areas.

In an embodiment of the present disclosure, the pain monitoring application 108 may be used in various fields including dental treatment, medical treatment, sports/athletics, occupational health and the like. For example, a dental hygienist may utilize the pain monitoring application 108 to assist cleaning of patient's teeth.

In an embodiment of the present disclosure, the communication device 202 is associated with the one or more sensors 104 via the network (for e.g., LAN, WAN, MAN, Bluetooth, Wi-Fi and the like). Thus, the communication device 202 collects the inputs relating to the autonomic reactions including the heart rate variability, the galvanic skin response, the amount of pressure applied and the like from the user 102.

Going further, the communication device 202 includes any suitable type of portable electronic device. Examples of the communication device 202 include but may not be limited to a personal e-mail device (e.g., a Blackberry™ made available by Research in Motion of Waterloo, Ontario), a personal data assistant (“PDA”), a cellular telephone, a Smartphone, a handheld gaming device, a digital camera, the laptop computer, and a tablet computer. In another embodiment of the present disclosure, the communication device 202 can be a desktop computer.

From the perspective of this disclosure, the control circuitry module 206 includes any processing circuitry or processor operative to control the operations and performance of the communication device 202. For example, the control circuitry module 206 may be used to run operating system applications, firmware applications, media playback applications, media editing applications, or any other application. In an embodiment, the control circuitry module 206 drives a display and process inputs received from a user interface.

From the perspective of this disclosure, the storage module 208 includes one or more storage mediums including a hard-drive, solid state drive, flash memory, permanent memory such as ROM, any other suitable type of storage component, or any combination thereof. The storage module 208 may store, for example, media data (e.g., music and video files), application data (e.g., for implementing functions on the communication device 202).

From the perspective of this disclosure, the input/output circuitry module 210 may be operative to convert (and encode/decode, if necessary) analog signals and other signals into digital data. In an embodiment, the input/output circuitry module 210 may also convert the digital data into any other type of signal and vice-versa. For example, the input/output circuitry module 210 may receive and convert physical contact inputs (e.g., from a multi-touch screen), physical movements (e.g., from a mouse or sensor), analog audio signals (e.g., from a microphone), or any other input. The digital data may be provided to and received from the control circuitry module 206, the storage module 208 or any other component of the communication device 202.

It may be noted that the input/output circuitry module 210 is illustrated in FIG. 2 as a single component of the communication device 202; however those skilled in the art would appreciate that several instances of the input/output circuitry module 210 may be included in the communication device 202.

The communication device 202 may include any suitable interface or component for allowing the user 102 to provide inputs to the input/output circuitry module 210. The communication device 202 may include any suitable input mechanism. Examples of the input mechanism include but may not be limited to a button, keypad, dial, a click wheel, and a touch screen. In an embodiment, the communication device 202 may include a capacitive sensing mechanism, or a multi-touch capacitive sensing mechanism.

In an embodiment, the communication device 202 may include specialized output circuitry associated with output devices such as, for example, one or more audio outputs. The audio output may include one or more speakers built into the communication device 202, or an audio component that may be remotely coupled to the communication device 202.

The one or more speakers can be mono speakers, stereo speakers, or a combination of both. The audio component can be a headset, headphones or ear buds that may be coupled to the communication device 202 with a wire or wirelessly.

In an embodiment, the input/output circuitry module 210 may include display circuitry for providing a display visible to the user 102. For example, the display circuitry may include a screen (e.g., an LCD screen) that is incorporated in the communication device 202.

The display circuitry may include a movable display or a projecting system for providing a display of content on a surface remote from the communication device 202 (e.g., a video projector). In an embodiment, the display circuitry may include a coder/decoder to convert digital media data into the analog signals. For example, the display circuitry may include video Codecs, audio Codecs, or any other suitable type of Codec.

The display circuitry may include display driver circuitry, circuitry for driving display drivers or both. The display circuitry may be operative to display content. The display content can include media playback information, application screens for applications implemented on the electronic device, information regarding ongoing communications operations, information regarding incoming communications requests, or device operation screens under the direction of the control circuitry module 206. Alternatively, the display circuitry may be operative to provide instructions to a remote display.

In addition, the communication device 202 includes the communication circuitry module 212. The communication circuitry module 212 may include any suitable communication circuitry operative to connect to a communication network and to transmit communications (e.g., voice or data) from the communication device 202 to other devices within the communications network. The communication circuitry module 212 may be operative to interface with the communication network using any suitable communication protocol. Examples of the communication protocol include but may not be limited to Wi-Fi, Bluetooth RTM, radio frequency systems, infrared, LTE, GSM, GSM plus EDGE, CDMA, and quadband.

In an embodiment, the communication circuitry module 212 may be operative to create a communications network using any suitable communications protocol. For example, the communication circuitry module 212 may create a short-range communication network using a short-range communications protocol to connect to other devices. For example, the communication circuitry module 212 may be operative to create a local communication network using the Bluetooth, RTM protocol to couple the communication device 202 with a Bluetooth, RTM headset.

It may be noted that the computing device is shown to have only one communication operation; however, those skilled in the art would appreciate that the communication device 202 may include one more instances of the communication circuitry module 212 for simultaneously performing several communication operations using different communication networks. For example, the communication device 202 may include a first instance of the communication circuitry module 212 for communicating over a cellular network, and a second instance of the communication circuitry module 212 for communicating over Wi-Fi or using Bluetooth RTM.

In an embodiment, the same instance of the communication circuitry module 212 may be operative to provide for communications over several communication networks. In an embodiment, the communication device 202 may be coupled a host device for data transfers, synching the communication device 202, software or firmware updates, providing performance information to a remote source (e.g., providing riding characteristics to a remote server) or performing any other suitable operation that may require the communication device 202 to be coupled to a host device. Several computing devices may be coupled to a single host device using the host device as a server. Alternatively or additionally, the communication device 202 may be coupled to the several host devices (e.g., for each of the plurality of the host devices to serve as a backup for data stored in the communication device 202).

It may be noted that in FIG. 2, various modules of the pain monitoring application 108 are shown that illustrates the working of the pain monitoring application 108; however those skilled in the art would appreciate that the pain monitoring application 108 may have more number of modules that could illustrate overall functioning of the pain monitoring application 108.

It may be noted that in FIG. 2, the pain monitoring application 108 includes the associating engine 204a, the tracking module 204b, the generating module 204c, the treatment module 204d and the database 204e for treating the pain of the user 102; however those skilled in the art would appreciate that the pain monitoring application 108 may include more modules that could treat the pain of the user 102.

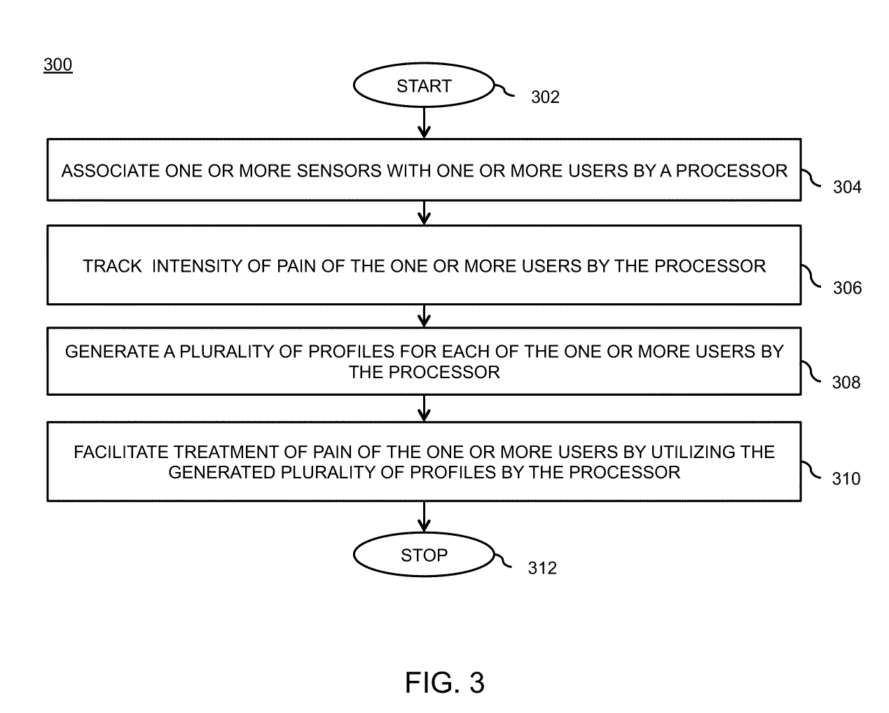

FIG. 3 is a flowchart 300 for treating the pain of users, in accordance with the various embodiments of the present disclosure. The flowchart 300 initiates at step 302. At step 304, the associating engine 204a associates the one or more sensors 104 with the user 102. The one or more sensors 104 measure the one or more bio-markers associated with the user 102 in the real time. At step 306, the tracking module 204b tracks the intensity of pain of the user 102. The tracking is performed by enabling the interaction of the one or more devices 112 with the user 102. At step 308, the generating module 204c generates the plurality of profiles for the user 102. At step 310, the treatment module 204d facilitates the treatment of the pain of the user 102 by utilizing the plurality of profiles. The facilitating of the treatment is based on the one or more pre-determined criteria (as discussed earlier). The flowchart 300 terminates at step 312.

It may be noted that the flowchart 300 is explained to have above stated process steps; however, those skilled in the art would appreciate that the flowchart 300 may have more/less number of process steps which may enable all the above stated embodiments of the present disclosure.

The present disclosure provides various advantages over the prior art. The present disclosure lets one know the effect of the treatment being given to the patient is as per the expectations of the physician/nurse or any person giving the treatment. A pre-defined threshold can be set of the expected improvement in the biomarkers. If the pre-defined threshold is not reached (even the lower range of the expected benefit in the given case), changes/adjustments in the treatment are performed accordingly. In addition, it enables the patient to track if the treatment of the doctor/physician is actually working for him or not. If not, the patient can decide the further proceedings.

While the disclosure has been presented with respect to certain specific embodiments, it will be appreciated that many modifications and changes may be made by those skilled in the art without departing from the spirit and scope of the disclosure. It is intended, therefore, by the appended claims to cover all such modifications and changes as fall within the true spirit and scope of the disclosure.

What is claimed is:

1. A computer-implemented method for treating pain of one or more users, the method comprising:
associating, with a processor, one or more sensors with the one or more users, wherein the one or more sensors measure one or more bio-markers associated with the one or more users in real time;
tracking, with the processor, intensity of pain of the one or more users, wherein the tracking being performed by enabling interaction of one or more devices with the one or more users;
generating, with the processor, a plurality of profiles for each of the one or more users, wherein the plurality of profiles being generated by the associating and the tracking of the intensity of the pain of the one or more users; and
facilitating, with the processor, treatment of the pain of the one or more users by utilizing the generated plurality of profiles, and wherein the facilitating being based on one or more pre-determined criteria.

2. The computer-implemented method as recited in claim 1, further comprising storing, with the processor, the one or more bio-markers associated with each of the one or more users and the generated plurality of profiles.

3. The computer-implemented method as recited in claim 1, wherein the plurality of profiles comprises at least one of one or more pain profiles, one or more side-effect profiles and one or more treatment profiles.

4. The computer-implemented method as recited in claim 1, wherein the one or more sensors comprises at least one of a ballistocardiogram, an impedance cardiography, a dispersion based electrocardiography, a respiration sensor and an emotion detector.

5. The computer-implemented method as recited in claim 1, wherein the one or more bio-markers associated with each of the one or more users comprises at least one of heart rate (HR), heart rate variability (HRV), skin conductance, respiration information, blood pressure, photoplethysmography (PPG), oxygen saturation, electrocardiography (ECG), electroencephalography (EEG), muscle activity (EMG) and restlessness.

6. The computer-implemented method as recited in claim 1, wherein the one or more devices comprises at least one of one or more transcutaneous electrical nerve simulation devices, one or more acupuncture devices, one or more devices providing ultrasound based pain relief and one or more spinal stimulators.

7. The computer-implemented method as recited in claim 1, further comprising assessing, with the processor, the intensity of the pain of the one or more users by viewing the intensity of the pain on displays of the one or more devices.

8. The computer-implemented method as recited in claim 1, wherein a pre-determined criterion of the one or more pre-determined criteria comprises throbbing of pain area on body of each of the one or more users, the throbbing enables application of symmetrical pulses of fixed frequency to reduce the pain.

9. The computer-implemented method as recited in claim 1, wherein a pre-determined criterion of the one or more pre-determined criteria comprises application of at least one of heat, pressure and laser light on the pain area on the body of each of the one or more users.

10. The computer-implemented method as recited in claim 1, wherein a pre-determined criterion of the one or more pre-determined criteria comprises electromagnetically heating of the pain area on the body of each of the one or more users by utilizing ultrasonic radiations.

11. A computer program product comprising a non-transitory computer readable medium storing a computer readable program, wherein the computer readable program when executed on a computer causes the computer to perform steps comprising:
associating one or more sensors with one or more users, wherein the one or more sensors measure one or more bio-markers associated with the one or more users in real time;
tracking intensity of pain of the one or more users, wherein the tracking being performed by enabling interaction of one or more devices with the one or more users;
generating a plurality of profiles for each of the one or more users, wherein the plurality of profiles being generated by the associating and the tracking of the intensity of the pain of the one or more users; and
facilitating treatment of the pain of the one or more users by utilizing the generated plurality of profiles, and wherein the facilitating being based on one or more pre-determined criteria.

12. The computer program product as recited in claim 11, wherein the computer readable program when executed on the computer causes the computer to perform a further step of storing the one or more bio-markers associated with each of the one or more users and the generated plurality of profiles.

13. A system for treating pain of one or more users, the system comprising:
one or more devices, the one or more devices being configured to treat the pain of the one or more users, wherein the one or more devices comprises at least one of one or more transcutaneous electrical nerve simulation devices, one or more acupuncture devices, one or more devices providing ultrasound based pain relief and one or more spinal stimulators; and
a communication device, wherein the communication device further comprises a pain monitoring application to monitor and treat the pain experienced by the one or more users. The pain monitoring application further comprises:
an associating engine in a processor, the associating engine being configured to associate one or more sensors with the one or more users, wherein the one or more sensors measure one or more bio-markers associated with the one or more users in real time;
a tracking module in the processor, the tracking module being configured to track intensity of pain of the one or more users, wherein the tracking being performed by enabling interaction of the one or more devices with the one or more users;
a generating module in the processor, the generating module being configured to generate a plurality of profiles for each of the one or more users, wherein the plurality of profiles being generated by the associating and the tracking of the intensity of the pain of the one or more users;
a treatment module in the processor, the treatment module being configured to facilitate treatment of the pain of the one or more users by utilizing the plurality of profiles, wherein the facilitating being based on one or more pre-determined criteria; and
a database in the processor, the database being configured to store the one or more bio-markers associated with each of the one or more users and the generated plurality of profiles.

14. The system as recited in claim 13, further comprising an application server being configured to run the pain monitoring application.

15. The system as recited in claim 13, wherein the plurality of profiles comprises at least one of one or more pain profiles, one or more side-effect profiles and one or more treatment profiles.

16. The system as recited in claim 13, wherein the one or more sensors comprises at least one of a ballistocardiogram, an impedance cardiography, a dispersion based electrocardiography, a respiration sensor and an emotion detector.

17. The system as recited in claim 13, wherein the one or more bio-markers associated with each of the one or more users comprises at least one of heart rate (HR), heart rate variability (HRV), skin conductance, respiration information, blood pressure, photoplethysmography (PPG), oxygen saturation, electrocardiography (ECG), electroencephalography (EEG), muscle activity (EMG) and restlessness.

18. The system as recited in claim 13, wherein a pre-determined criterion of the one or more pre-determined criteria comprises throbbing of pain area on body of each of the one or more users, the throbbing enables application of symmetrical pulses of fixed frequency to reduce the pain.

19. The system as recited in claim 13, wherein a pre-determined criterion of the one or more pre-determined criteria comprises application of at least one of heat, pressure and laser light on the pain area on the body of each of the one or more users.

20. The system as recited in claim 13, wherein a pre-determined criterion of the one or more pre-determined criteria comprises electromagnetically heating of the pain area on the body of each of the one or more users by utilizing ultrasonic radiations.
